(19) United States
(12) Patent Application Publication
(10) Pub. No.: US 2026/0274956 A1
(43) Pub. Date: Sep. 17, 2026

(54) METHODS OF PURIFYING ANTI-PD-1 ANTIBODIES OR ANTIGEN-BINDING FRAGMENTS THEREOF USING MIXED-MODE ANION-EXCHANGE CHROMATOGRAPHY

(71) Applicant: Merck Sharp & Dohme LLC, Rahway, NJ (US)

(72) Inventors: Mark A. BROWER, Bound Brook, NJ (US); Nuno Jorge DOS SANTOS PINTO, Westfield, NJ (US); William J. RAYFIELD, High Bridge, NJ (US); Julie R. ROBINSON, Garwood, NJ (US)

(73) Assignee: Merck Sharp & Dohme LLC, Rahway, NJ (US)

(21) Appl. No.: 19/674,728

(22) Filed: May 12, 2026

Related U.S. Application Data
(63) Continuation of application No. PCT/US2024/059143, filed on Dec. 9, 2024.
(60) Provisional application No. 63/609,155, filed on Dec. 12, 2023.

(51) Int. Cl. C07K 16/28; C07K 1/18

(52) U.S. Cl. CPC C07K 16/2818; C07K 1/18; C07K 2317/24

(57) ABSTRACT

Provided herein are methods of separating host cell proteins from anti-PD-1 antibodies or antigen binding fragments in chromatographic processes.

### CROSS-REFERENCE TO RELATED APPLICATIONS

This application is a continuation of PCT application No. PCT/US2024/059143, filed Dec. 9, 2024, which claims priority from U.S. Provisional Application No. 63/609,155, filed Dec. 12, 2023, each of which is incorporated herein by reference in its entirety.

### REFERENCE TO SEQUENCE LISTING SUBMITTED ELECTRONICALLY

This application contains an electronic Sequence Listing which has been submitted in XML file format via Patent Center, the content of which is incorporated by reference in its entirety. The Sequence Listing XML file submitted via Patent Center is entitled “25871-US-CNT_SL.XML”, with a size of 18 bytes; and a date of creation of Apr. 5, 2024.

### FIELD OF THE INVENTION

Provided herein are methods of separating host cell proteins (HCP) (e.g., lipases) from anti-PD-1 antibodies in chromatographic processes.

### BACKGROUND OF THE INVENTION

Immune checkpoint therapies targeting the programmed death receptor-1 (PD-1) axis have resulted in groundbreaking improvements in clinical response in multiple human cancers (Brahmer et al., N Engl J Med 2012, 366:2455-65; Garon et al. N Engl J Med 2015, 372:2018-28; Hamid et al., N Engl J Med 2013, 369:134-44; Robert et al., Lancet 2014, 384:1109-17; Robert et al., N Engl J Med 2015, 372:2521-32; Robert et al., N Engl J Med 2015, 372:320-30; Topalian et al., N Engl J Med 2012, 366:2443-54; Topalian et al., J Clin Oncol 2014, 32:1020-30; Wolchok et al., N Engl J Med 2013, 369:122-33). The interaction of the PD-1 receptor on T-cells with its ligands, PD-L1 and PD-L2, on tumor and immune infiltrating cells regulates T-cell mediated immune responses and may play a role in immune escape by human tumors (Pardoll DM. Nat Rev Cancer 2012, 12:252-64). Binding of PD-1 to either of its ligands results in delivery of an inhibitory stimulus to the T cell. Immune therapies targeting the PD-1 axis include monoclonal antibodies directed to the PD-1 receptor (KEYTRUDA® (pembrolizumab), Merck and Co., Inc., Rahway, NJ and OPDIVO® (nivolumab), Bristol-Myers Squibb, Princeton, NJ) and also those that bind to the PD-L1 ligand (MPDL3280A; TECENTRIQ® (atezolizumab), Genentech, San Francisco, CA). Both therapeutic approaches have demonstrated anti-tumor effects in numerous cancer types.

In bioprocessing and manufacturing of antibodies (e.g., monoclonal antibodies), host cell proteins (HCP) (e.g., lipases) constitute impurities that are often difficult to remove from the antibodies. Such impurities can cause various issues in the safety and efficacy of biopharmaceuticals. Regulatory agencies throughout the world require that biopharmaceutical products meet certain acceptance criteria, including a certain level of impurities and tests for detection and quantification of impurities. Several anti-PD-1 antibodies are in clinical development, and it is desirable to develop efficient and effective processes to remove HCP (e.g., lipases) from these antibodies.

### SUMMARY OF THE INVENTION

The present disclosure provides methods of separating HCP (e.g., lipases) from a composition comprising anti-PD-1 antibodies or antigen-binding fragments through mixed-mode anion exchange (AEX) chromatographic processes comprising the steps of:
(a) passing a load fluid comprising the composition through the mixed-mode anion exchange resin under a loading operating condition; and
(b) collecting the anti-PD-1 antibodies or antigen-binding fragments in a flowthrough.

In one embodiment, the lipase is PLBL2. In another embodiment, the conductivity of the loading operating condition is 2-50 mS/cm.

The present disclosure also provides a method of separating a host cell lipase from a composition comprising anti-PD-1 antibodies comprising main species, acidic species, basic species, and a host cell lipase, wherein the host cell lipase is separated using a mixed-mode anion exchange (AEX) chromatographic process. The disclosure also provides a method of purifying anti-PD-1 antibody main species from a composition comprising anti-PD-1 antibodies comprising main species, acidic species, and basic species through a mixed-mode anion exchange (AEX) chromatographic process.

The present disclosure also provides a method of controlling the load challenge of a protein in a chromatographic process.

### BRIEF DESCRIPTION OF THE DRAWINGS

FIG. 1. Impact of conductivity (abscissa), pH (greyscale), and load (marker size) on PLBL2 levels (ordinate) for Poros 50 HS CEX resin.

FIG. 2. Contour plot of PLBL2 levels on Capto MMC Mixed-Mode Cation Ion Exchange (MMCEX) resin as a function of salt concentration and pH.

FIG. 3. Contour plot of PLBL2 levels on Capto Adhere Mixed-Mode Anion Exchange (MMAEX) as a function of conductivity and pH at loadings between 200 g/L to 300 g/L.

FIG. 4. Yield contour plot for Capto Adhere MMAEX optimization runs as a function of conductivity and pH at 400 g/L loading.

FIG. 5. Yield data by fraction for Capto Adhere MMAEX optimization runs at pH 6.2-7.2, 4-15 mS/cm up to 400 g/L load. The x-axis represents a unique set of operating conditions (run #) as listed in Table A.

TABLE A

| Run | pH | Cond. (mS/cm) |
|---|---|---|
| 1 | 6.2 | 2 |
| 2 | 6.4 | 2 |
| 3 | 6.7 | 2 |
| 4 | 7 | 2 |
| 5 | 7.2 | 2 |
| 6 | 7 | 5 |
| 7 | 6.2 | 8.5 |
| 8 | 6.7 | 8.5 |
| 9 | 6.7 | 8.5 |
| 10 | 7.2 | 8.5 |
| 11 | 6.4 | 10 |
| 12 | 7 | 10 |
| 13 | 6.2 | 15 |
| 14 | 6.4 | 15 |
| 15 | 6.7 | 15 |
| 16 | 7.2 | 15 |

FIG. 6A-B High Molecular Weight (HMW) levels in Capto Adhere MMAEX flow through for (A) product pool as a function of conductivity and pH at 400 g/L load and (B) product fractions. The x-axis represents a unique set of operating conditions (run #) as listed in Table A.

FIG. 7A-B PLBL2 levels in (A) Capto Adhere MMAEX flow through pool as a function of conductivity and pH at 400 g/L load and (B) product fractions at 200 g/L load and 400 g/L load (pool). The x-axis represents a unique set of operating conditions (run #) as listed in Table A.

FIG. 8A-B PLBL2 levels in Capto Adhere MMAEX flowthrough as a function of (A) conductivity and (B) pH. (A) shows trend line for correlation between conductivity and PLBL2 values with R² and root mean square error (RMSE). No correlation was observed between pH and PLBL2 values.

FIG. 9A-B (A) Yield and (B) HMW results for Capto Adhere optimization screen under Example 3. The x-axis represents a unique set of operating conditions (run #) as listed in Table B. Note that similar yield trends were observed across the pH/conductivity space.

TABLE B

| | | Conductivity |
|---|---|---|
| Run | pH | (mS/cm) |
| 1 | 6.8 | 20 |
| 2 | 7.0 | 20 |
| 3 | 7.2 | 20 |
| 4 | 7.4 | 20 |
| 5 | 7.4 | 15 |
| 6 | 7.0 | 12 |
| 7 | 7.2 | 12 |
| 8 | 7.4 | 12 |

FIG. 10: Bench scale (4 mL) evaluation of Capto Adhere MM AEX performance at 8 mS/cm and 14 mS/cm. Yield, HMW, Host Cell Protein (HCP), residual Protein A material (rPROA), PLBL2 levels affected by load challenge and conductivity. LOQ refers to Limit of Quantification.

FIG. 11. Comparison of yield and Basic 1 species through use of dynamic loading compared to no dynamic loading on the Capto Adhere MMAEX flowthrough polishing chromatography step.

FIG. 12: Ion exchange chromatography analytical diagram of pembrolizumab after Anion exchange chromatography and Protein A chromatography purification of HCCF from a continuous perfusion process. The retention time of each peak is annotated in the figure.

### DETAILED DESCRIPTION OF THE INVENTION

### Definitions

Certain technical and scientific terms are specifically defined below. Unless specifically defined elsewhere in this document, all other technical and scientific terms used herein have the meaning commonly understood by one of ordinary skill in the art to which this disclosure relates. In case of conflict, the present specification, including definitions, will control.

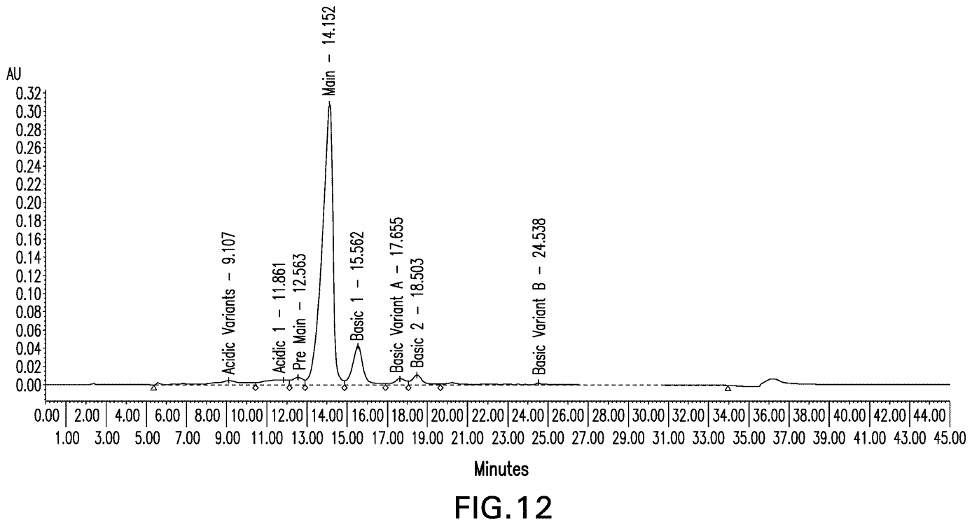

As used herein, “acidic species” refers to the anti-PD-1 antibody species that is more acidic (e.g., as determined by cation exchange chromatography) than the anti-PD-1 antibody main species. Such acidic species are detected by various chromatography purification methods for separating molecule variants by charge, such as ion exchange, for example, cation exchange chromatography (e.g., the method described in Example 6) or WCX-10 HPLC (a weak cation exchange chromatography), optionally followed by mass spectroscopy. Generally, the acidic species has a lower isoelectric point (pI) than the main species, and can have a more acidic character due to for example, methionine oxidation, sialylation of asparagine residues or deamidated variants of the antibody, or a combination thereof. Examples of the acidic species include but are not limited to the acidic variants, acidic 1 and pre-main peaks identified in FIG. 12. Any of the acidic species may also have one or more of CHO N-linked glycans selected from the group consisting of G0-F, G1-F, G2-F, G0, G1, G2 and Man5, for example at N297 in the CH2 domain.

In one embodiment, the anti-PD-1 antibody acidic species is as identified by peak(s) eluted prior to the main peak according to a cation ion exchange method. In another embodiment, the anti-PD-1 antibody acidic species is as identified by peak(s) eluted prior to the main peak according to a weak cation ion exchange method. In an ion exchange chromatography method, the “% acidic species” refers to the total area of acidic species peaks divided by the total area of all peaks in the elution chromatogram.

As used herein, “acidic1 species” refers to an acidic species with the presence of one or more of deamidation, succinimide, aspartate or isoaspartate formation in one or more of N384, N389 and N390 of the heavy chain of the anti-PD-1 antibody main species. Such acidic1 species are detected by various chromatography purification methods for separating molecule variants by charge, such as ion exchange, for example, cation exchange chromatography (e.g., the method described in Example 6) or WCX-10 HPLC (a weak cation exchange chromatography), followed by mass spectroscopy of the acidic species peaks.

In one embodiment, the anti-PD-1 antibody acidic1 species is as identified in acidic 1 peak of FIG. 12, and eluted according to the cation ion exchange method described in Example 6. In an ion exchange chromatography method, the “% acidic1 species” refers to the total area of acidic 1 peak divided by the total area of all peaks in the elution chromatogram.

As used herein, “acidic variants species” refers to an acidic species with the presence of one or more of deamidation, succinimide, aspartate or isoaspartate formation in one or more of N31, N52, N55, N59, and N61 of the heavy chain; or the presence of M105 oxidation in the heavy chain; or a combination thereof of the anti-PD-1 antibody main species. Such acidic variants species are detected by various chromatography purification methods for separating molecule variants by charge, such as ion exchange, for example, cation exchange chromatography (e.g., the method described in Example 6) or WCX-10 HPLC (a weak cation exchange chromatography), followed by mass spectroscopy of the acidic species peaks.

In one embodiment, the anti-PD-1 antibody acidic variants species is the anti-PD-1 antibody species as identified by the acidic variants peak(s) in FIG. 12, and eluted according to the cation ion exchange method described in Example 6. In an ion exchange chromatography method, the “% acidic variants species” refers to the total area of acidic variants peak(s) divided by the total area of all peaks in the elution chromatogram.

As used herein, “basic species” refers to the anti-PD-1 antibody species that is more basic (e.g., as determined by cation exchange chromatography) than the anti-PD-1 antibody main species. Such basic species are detected by various chromatography purification methods for separating molecule variants by charge, such as ion exchange, for example, cation exchange chromatography (e.g., the method described in Example 6) or WCX-10 HPLC (a weak cation exchange chromatography), optionally followed by mass spectroscopy. Generally, the basic species has a higher pI than the main species, and can have a more basic character due to modifications or differences from the main species including but not limited to the presence of the C-terminal lysine residue (SEQ ID NO:10 or 12), the presence of N-terminal glutamine residue (SEQ ID NO: 10 or 13), or alpha-amidation of a C-terminal leucine residue (SEQ ID NO: 14 or 15), truncation of N-terminal amino acid residues in one or both heavy chains according to the amino acid sequence in any one of SEQ ID NO: 10-15, or a combination thereof. Examples of the basic species include but are not limited to the basic variant A, basic variant B, basic 1 and basic 2 peaks identified in FIG. 12. Any of the basic species may also have one or more of CHO N-linked glycans selected from the group consisting of G0-F, G1-F, G2-F, G0, G1, G2 and Man5, for example at N297 in the CH2 domain.

In one embodiment, the main species comprises the anti-PD-1 antibody consisting of two heavy chains and two light chains, each heavy chain consisting of the amino acid sequence of SEQ ID NO: 11, and each light chain consisting of the amino acid sequence of SEQ ID NO: 5. In another embodiment, the anti-PD-1 antibody main species is produced from a Chinese Ovary cell that comprises a polynucleotide encoding a light chain that consists of the amino acid sequence of SEQ ID NO: 5 and a polynucleotide encoding a heavy chain that consists of the amino acid sequence of SEQ ID NO: 10, 13 or 15, or a polynucleotide encoding the light chain and the heavy chain.

In one embodiment, the main species is identified as the main peak according to a cation ion exchange method. In an ion exchange method, the “% main species” refers to the total area of main peak divided by the total area of all peaks in the elution chromatogram.

As used herein, “basic 1 species” refers to a basic species consisting of two heavy chains and two light chains, one heavy chain consisting of the amino acid sequence of SEQ ID NO: 11, one heavy chain consisting of the amino acid sequence of SEQ ID NO: 12, and each light chain consisting of the amino acid sequence of SEQ ID NO: 5; or a basic species consisting of two heavy chains and two light chains, one heavy chain consisting of the amino acid sequence of SEQ ID NO: 11, one heavy chain consisting of the amino acid sequence of SEQ ID NO: 14, wherein the C-terminal leucine is alpha-amidated, and each light chain consisting of the amino acid sequence of SEQ ID NO: 5; or a combination thereof. Such basic 1 species are detected by various chromatography purification methods for separating molecule variants by charge, such as ion exchange, for example, cation exchange chromatography (e.g., the method described in Example 6) or WCX-10 HPLC (a weak cation exchange chromatography), followed by mass spectroscopy of the basic species peaks.

In one embodiment, the anti-PD-1 antibody basic 1 species is as identified by basic 1 peak in FIG. 12, and eluted according to the cation ion exchange method described in Example 6. In an ion exchange chromatography method, the “% basic 1 species” refers to the total area of basic 1 peak divided by the total area of all peaks in the elution chromatogram.

As used herein, “deamidated variant” refers to an antibody wherein one or more asparagine residue(s) have been deamidated. The deamidated variant can be in the form of succinimide, aspartate or isoaspartate. i.e., the neutral amide side chain has been converted to a residue with an overall acidic character.

In one aspect of measuring the main species, acidic species or basic species, a Thermo Scientific ProPac WCX-10 column is used for the cation ion exchange method. In another embodiment, a Thermo Scientific ProPac WCX-10 column is used, with a Mobile Phase (A) 24 mM MES pH 6.1 with 4% acetonitrile, and mobile phase (B) 20 mM sodium phosphate, 95 mM NaCl pH 8.0 with 4% acetonitrile, and a column temperature of 35° C. In one embodiment, a non-linear gradient is used with: 22%-22% B for 0-0.6 min; 22%-29% B for 0.6-15.0 min; 29%-70% B for 15.0-30.0 min; 70%-100% B for 30.0-30.5 min; and 100%-100% B from 30.5-33.0 min. In a further embodiment, the cation ion exchange method is described in Example 6.

The term “load challenge” as used herein, refers to the amount of protein (in grams) loaded onto the resin (per Liter) in the chromatography step.

The term “operating condition,” “operation condition,” “processing condition,” or “process condition,” as used interchangeably herein, refers to the condition for operating a chromatographic process. The operating condition can be equilibration condition, loading condition, wash condition, and/or elution condition, etc. The operating condition includes but is not limited to the type of the chromatographic resin, the resin backbone, the resin ligand, the pH of the operating solution, the composition of the operating solution, the concentration of each ingredient of the operating solution, the conductivity of the operating solution, the ionic strength of the operating solution, the cationic strength of the operating solution, the anionic strength of the operating solution, or a combination of two or more above factors.

The term “operating solution” refers to the solution used in operating a chromatographic process. The operating solution can be equilibration solution, loading or feed solution, wash solution, and/or elution solution, etc.

“Flowthrough,” as used herein, refers to the liquid from the loading fluid that passes through a chromatography resin.

The term “about”, when modifying the quantity (e.g., mM, or M) of a substance or composition, the pH of a solution, or the value of a parameter characterizing a step in a method, or the like refers to variation in the numerical quantity that can occur, for example, through typical measuring, handling and sampling procedures involved in the preparation, characterization and/or use of the substance or composition; through instrumental error in these procedures; through differences in the manufacture, source, or purity of the ingredients employed to make or use the compositions or carry out the procedures; and the like, and is up to 10% of the value. In certain embodiments, “about” can mean a variation of +0.1%, 0.5%, 1%, 2%, 3%, 4%, 5%, or 10% of the value.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, unless otherwise indicated, “antibody fragment” or “antigen binding fragment” refers to antigen binding fragments of antibodies, i.e., antibody fragments that retain the ability to bind specifically to the antigen bound by the full-length antibody, e.g., fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified method, or composition. As a non-limiting example, anti-PD-1 antibodies or antigen binding fragments that consist essentially of a recited amino acid sequence may also include one or more amino acids, including substitutions of one or more amino acid residues, which do not materially affect the properties of the binding compound.

“Framework region” or “FR” as used herein means the immunoglobulin variable regions excluding the CDR regions.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Mixed-mode” as used herein refers to chromatography with ligands that interact with protein(s) in the sample through multiple intermolecular interactions (e.g., affinity, size exclusion, hydrogen bonding, thiophilic, hydrophilic, hydrophobic, and/or ionic). In some embodiments, the mixed-mode chromatography resin provides ligands that interact with the protein(s) through intermolecular interactions of hydrogen bonding, thiophilic, hydrophilic, hydrophobic, and/or ionic interaction. In other embodiments, the mixed-mode chromatography resin provides ligands that interact with the protein(s) through intermolecular interactions of hydrogen bonding, thiophilic, hydrophobic, and/or ionic interaction. In other embodiments, the mixed-mode chromatography resin provides ligands that interact with the protein(s) through intermolecular interactions of hydrophobic, and/or ionic interaction.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example. See also Presta (2005) J. Allergy Clin. Immunol. 116:731.

“Pembrolizumab” (formerly known as MK-3475, SCH 900475 and lambrolizumab) alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 2. Pembrolizumab has been approved by the U.S. FDA as described in the Prescribing Information for KEYTRUDA™ (Merck & Co., Inc., Rahway, NJ USA; initial U.S. approval 2014).

As used herein, a “pembrolizumab variant” means a monoclonal antibody that comprises heavy chain and light chain sequences that are substantially identical to those in pembrolizumab, except for having three, two or one conservative amino acid substitutions in the light chain at positions that are located outside of the light chain CDRs and/or six, five, four, three, two or one conservative amino acid substitutions in the heavy chain that are located outside of the heavy chain CDRs, e.g., the variant positions are located in the FR regions or the constant region. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

As used herein, including the appended claims, the singular forms of words such as “a,” “an,” and “the,” include their corresponding plural references unless the context clearly dictates otherwise. Unless otherwise required by context, singular terms shall include pluralities and plural terms shall include the singular.

As used herein, the terms “at least one” item or “one or more” item each include a single item selected from the list as well as mixtures of two or more items selected from the list.

Any example(s) following the term “e.g.” or “for example” is not meant to be exhaustive or limiting.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. All ranges also are intended to include all included sub-ranges, although not necessarily explicitly set forth. For example, a range of pH 4.0-5.0 is intended to include pH 4.0, 4.1, 4.13, 4.2, 4.1-4.6, 4.3-4.4, and 5.0. In addition, the term “or,” as used herein, denotes alternatives that may, where appropriate, be combined; that is, the term “or” includes each listed alternative separately as well as their combination.

Exemplary methods and materials are described herein, although methods and materials similar or equivalent to those described herein can also be used in the practice or testing of the present disclosure. The materials, methods, and examples are illustrative only and not intended to be limiting.

### I. Anti-PD-1 Antibodies and Antigen-Binding Fragments Thereof

In some embodiments, anti-human PD-1 antibodies or antigen binding fragments thereof for use in the methods of the invention comprise a light chain variable region comprising three light chain CDRs of CDRL1, CDRL2 and CDRL3 and a heavy chain variable region comprising three heavy chain CDRs of CDRH1, CDRH2 and CDRH3.

In one embodiment of the invention, CDRL1 is SEQ ID NO:1, CDRL2 is SEQ ID NO: 2, and CDRL3 is SEQ ID NO:3. In one embodiment, CDRH1 is SEQ ID NO:6, CDRH2 is SEQ ID NO: 7, and CDRH3 is SEQ ID NO:8. In one embodiment, the three light chain CDRs 1, 2 and 3 are SEQ ID NO:1, SEQ ID NO:2, and SEQ ID NO:3, respectively, and the three heavy chain CDRs 1, 2 and 3 are SEQ ID NO:6, SEQ ID NO:7 and SEQ ID NO:8, respectively.

Anti-PD-1 binding fragments of the methods of the invention comprise a light chain variable region and a heavy chain variable region. In one embodiment of the methods of the invention, the antibodies or antigen binding fragments comprise a light chain variable region comprising or consisting of SEQ ID NO:4 and a heavy chain variable region comprising or consisting of SEQ ID NO:9.

In another embodiment, the methods of the invention comprise antibodies or antigen binding fragments that have a V_{L} domain and/or a V_{H} domain with at least 95%, 90%, 85%, 80%, 75% sequence homology to one of the V_{L} domains or V_{H} domains described above, and exhibit specific binding to PD-1. In another embodiment, the antibodies or antigen binding fragments of the methods of the invention comprise V_{L} and V_{H} domains having up to 1, 2, 3, 4, or 5 or more amino acid substitutions, and exhibit specific binding to PD-1.

In any of the embodiments above, the anti-PD-1 antibodies may be full-length anti-PD-1 antibodies that specifically bind human PD-1. In certain embodiments, the full-length anti-PD-1 antibodies are selected from any class of immunoglobulins, including IgM, IgG, IgD, IgA, and IgE. Preferably, the antibodies are IgG antibodies. Any isotype of IgG can be used, including IgG₁, IgG₂, IgG₃, and IgG₄. Different constant domains may be appended to the V_{L} and V_{H} regions provided herein. For example, if a particular intended use of the antibodies (or fragments) of the invention were to call for altered effector functions, a heavy chain constant domain other than IgG1 may be used. Although IgG1 antibodies provide for long half-life and effector functions, such as complement activation and antibody-dependent cellular cytotoxicity, such activities may not be desirable for all uses of the antibodies. In such instances an IgG4 constant domain, for example, may be used.

In embodiments of the invention, the anti-PD-1 antibodies comprise a light chain comprising or consisting of a sequence of amino acid residues as set forth in SEQ ID NO:5 and a heavy chain comprising or consisting of a sequence of amino acid residues as set forth in SEQ ID NO: 10. In some methods of the invention, the anti-PD-1 antibodies are pembrolizumab, or pembrolizumab variants. In other embodiments of the methods of the invention, the anti-human PD-1 antibodies consist of two light chains and two heavy chains, wherein the two light chains consist of the amino acid sequence set forth in SEQ ID NO:5, wherein the two heavy chains consist of the amino acid sequence set forth in any one of SEQ ID NO: 10-15, or a combination thereof.

Ordinarily, amino acid sequence variants of the anti-PD-1 antibodies and antigen binding fragments of the invention will have an amino acid sequence having at least 75% amino acid sequence identity with the amino acid sequence of a reference antibody or antigen binding fragment (e.g., heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the anti-PD-1 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

Likewise, either class of light chain can be used in the methods herein. Specifically, kappa, lambda, or variants thereof are useful in the present methods.

TABLE 2
Exemplary PD-1 Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYLHWY | 4 |
| Region | QQKPGQAPRLLIYLASYLESGVPARFSGSGSGTDFTLTISS | |
| | LEPEDFAVYYCQHSRDLPLTFGGGTKVEIK | |
| Light Chain | EIVLTQSPATLSLSPGERATLSCRASKGVSTSGYSYLHWY | 5 |
| | QQKPGQAPRLLIYLASYLESGVPARFSGSGSGTDFTLTISS | |
| | LEPEDFAVYYCQHSRDLPLTFGGGTKVEIKRTVAAPSVFI | |
| | FPPSDEQLKSGTASVVCLLNNFYPREAKVQWKVDNALQ | |
| | SGNSQESVTEQDSKDSTYSLSSTLTLSKADYEKHKVYAC | |
| | EVTHQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYWV | 9 |
| Region | RQAPGQGLEWMGGINPSNGGTNFNEKFKNRVTLTTDSST | |
| | TTAYMELKSLQFDDTAVYYCARRDYRFDMGFDYWGQG | |
| | TTVTVSS | |
| Heavy | QVQLVQSGVEVKKPGASVKVSCKASGYTFTNYYMYWV | 10 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLGK | |
| Heavy | XVQLVQSGVEVKKPGASVKVSCKASGYTFTNYYMYWV | 11 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLG | |
| | X is pyroglutamate | |
| Heavy | XVQLVQSGVEVKKPGASVKVSCKASGYTFTNYYMYWV | 12 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLGK | |
| | X is pyroglutamate | |
| Heavy | QVQLVQSGVEVKKPGASVKVSCKASGYTFTNYYMYWV | 13 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSLG | |
| Heavy | XVQLVQSGVEVKKPGASVKVSCKASGYTFTNYYMYWV | 14 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSL | |
| | X is pyroglutamate | |
| Heavy | QVQLVQSGVEVKKPGASVKVSCKASGYTFTNYYMYWV | 15 |
| Chain | RQAPGQGLEWMGGINPSNGGTNFNEKFKNRVTLTTDSST | |
| | TTAYMELKSLQFDDTAVYYCARRDYRFDMGFDYWGQG | |
| | TTVTVSSASTKGPSVFPLAPCSRSTSESTAALGCLVKDYF | |
| | PEPVTVSWNSGALTSGVHTFPAVLQSSGLYSLSSVVTVPS | |
| | SSLGTKTYTCNVDHKPSNTKVDKRVESKYGPPCPPCPAP | |
| | EFLGGPSVFLFPPKPKDTLMISRTPEVTCVVVDVSQEDPE | |
| | VQFNWYVDGVEVHNAKTKPREEQFNSTYRVVSVLTVLH | |
| | QDWLNGKEYKCKVSNKGLPSSIEKTISKAKGQPREPQVY | |
| | TLPPSQEEMTKNQVSLTCLVKGFYPSDIAVEWESNGQPE | |
| | NNYKTTPPVLDSDGSFFLYSRLTVDKSRWQEGNVFSCSV | |
| | MHEALHNHYTQKSLSLSL | |

### II. Chromatographic Processes

The chromatographic process for the separation of host cell lipase from the anti-PD-1 antibodies or antigen binding fragments can be a mixed-mode AEX chromatographic process. In one embodiment, the mixed-mode AEX chromatographic process is preceded by a protein A chromatography followed by AEX chromatography.

IEX chromatography separates molecules based on net charge of the molecules. Separation occurs as a result of competition between the charged molecule of interest and counter ions for oppositely charged ligand groups on the IEX chromatographic resin. Strength of the binding of the molecule to the IEX resin depends on the net charge of the molecules, which is affected by operating conditions, such as pH and ionic strength. AEX resins may contain substituents such as diethylaminoethyl (DEAE), trimethyalaminoethyl (TMAE), quaternary aminoethyl (QAE) and quaternary amine (O) groups. Cellulosic IEX resins such as DE23, DE32, DE52, CM-23, CM-32 and CM-52 are available from Whatman Ltd. Maidstone, Kent, U.K. Sephadex-based and cross-linked IEX resins are also known. For example, DEAE-, QAE-, CM-, and SP-Sephadex, and DEAE-, Q-, CM- and S-Sepharose, and Sepharose are all available from Cytiva, both DEAE and CM derived ethylene glycol-methacrylate copolymer such as TOYOPEARL™ DEAE-650S or M and TOYOPEARL™ CM-650S or M are available from Toso Haas Co., Philadelphia, PA. POROS™ HS, POROS™ HQ, POROS™ XS are available from Thermo Fisher Scientific, Waltham, MA.

Hydrophobic interaction chromatography (HIC) separates molecules based on hydrophobicity of molecules. Hydrophobic regions in the molecule of interest bind to the HIC resin through hydrophobic interaction. Strength of the interaction depends on operating conditions such as pH, ionic strength, and salt concentration. In general, HIC resins contain a base matrix (e.g., cross-linked agarose or synthetic copolymer material) to which hydrophobic ligands (e.g., alkyl or aryl groups) are coupled. Non-limiting examples of HIC resins include Phenyl SEPHAROSE™ 6 FAST FLOW™ (Pharmacia LKB Biotechnology, AB, Sweden); Phenyl SEPHAROSE™ High Performance (Pharmacia LKB Biotechnology, AB, Sweden); Octyl SEPHAROSE™ High Performance (Pharmacia LKB Biotechnology, AB, Sweden); Fractogel™ EMD Propyl or FRACTOGEL™ EMD Phenyl (E. Merck, Germany); MACRO-PREP™ Methyl or MACRO-PREP™ t-Butyl Supports (Bio-Rad, CA); WP HI-Propyl (C₃)™ (J. T. Baker, NJ); TOYOPEARL™ ether, phenyl or butyl (TosoHaas, PA); and Tosoh-Butyl-650M (Tosoh Corp., Tokyo, Japan).

Hydroxyapatite chromatography (HAC) uses an insoluble hydroxylated calcium phosphate of the formula [Ca₁₀(PO₄)₆(OH)₂] as both the matrix and the ligand. The functional groups of the HAC resin include pairs of positively charged calcium ions (C-sites) and negatively charged phosphate groups (P-sites). The C-sites can interact with carboxylate residues on the protein surface while the P-sites can interact with basic protein residues. Strength of the binding between the protein and the HAC resin depends on operating conditions including pH, ionic strength, composition of solution, concentration of each component of the composition, gradient of pH, gradient of component concentration, etc. Various HAC resins, such as CHT™ Ceramic Hydroxyapatite and CFT™ Ceramic Fluoroapatite, are commercially available.

Affinity chromatography separates molecules based on a highly specific interaction between the molecule of interest and the functional group of the resin, such as interaction between antigen and antibody, enzyme and substrate, receptor and ligand, or protein and nucleic acid, etc. Some commonly used affinity chromatographic resins include protein A or protein G resin to purify antibodies, avidin biotin resin to purify biotin/avidin and their derivatives, glutathione resin to purify glutathione S-transferase (GST)-tagged recombinant proteins, heparin resin to separate plasma coagulation proteins, IMAC resin to purify proteins that specifically interact with the metal ions, etc. Operating conditions of each affinity chromatography depend on the mechanism of the interaction and factors that affect the interaction. Commercial affinity chromatographic resins include but are not limited to MabSelect Sure, UNOsphere SUPrA™, Affi-Gel®, and Affi-Prep®

The mixed mode can be a combination of any two or more functions or mechanisms described above or understood by a person of ordinary skill in the art, such as a combination of IEX and HIC (e.g., AEX/HIC), a combination of AEX and CEX (AEX/CEX), or a combination of HIC, AEX, and CEX (HIC/AEX/CEX), etc. Exemplary mixed mode chromatographic resins include but are not limited to OminPac PCX-500, Primesep®, Obelisc R, Oblisc N, Acclaim Trinity P1, Acclaim Trinity P2, Capto Adhere, Capto Adhere Impres, Capto MMC, Capto MMC Impres, Capto Core 700, PPA Hypercel, HEA Hypercel, MEP Hypercel, Eshmuno HCX, Toyopearl MX-Trp-650M, Nuvia C Prime, CHT Type I, and CHT Type II. In some embodiments, the AEX mixed-mode chromatography resin provides ligands that interact with the proteins in the sample through hydrogen bonding, thiophilic, hydrophilic, and/or hydrophobic, and ionic intermolecular interactions. In other embodiments, the AEX mixed-mode chromatography resin provides ligands that interact with the proteins through hydrogen bonding, thiophilic, and/or hydrophobic, and anionic intermolecular interactions. In other embodiments, the AEX mixed-mode chromatography resin provides ligands that interact with the proteins through hydrophobic and ionic intermolecular interactions. In a preferred embodiment, the mixed-mode AEX comprises a positively charged aromatic compound. In a preferred embodiment, the mixed-mode AEX is Capto Adhere (from Cytiva). In another preferred embodiment, the mixed-mode AEX resin comprises N-benzyl-n-methyl ethanolamine attached to the resin. In a preferred embodiment, the mixed-mode AEX is Nuvia aPrime 4A (from BioRad). In another preferred embodiment, the mixed-mode AEX resin comprises

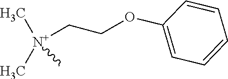

attached to the resin.

The present disclosure also provides a method of controlling the load challenge of a protein in a chromatographic process comprising the step of:
a) Loading the protein to a chromatographic resin in a load fluid;
b) Detecting the ultraviolet absorbance value of the load fluid;
c) Determining the load challenge from the absorbance value; and
d) Pausing the loading step when the load challenge reaches a target load challenge value.

The chromatographic resin can be any one of the foregoing resins. In one embodiment, the resin is mixed-mode AEX. The protein includes but is not limited to antibodies, antigen binding fragments, recombinant proteins, vaccine antigens, or Fc fusion proteins. In one embodiment, the protein is anti-PD-1 antibodies or antigen binding fragments described in Section I.

### III. Methods of Purifying Anti-PD-1 Antibodies or Antigen Binding Fragments Thereof

In one aspect, provided herein is a method of purifying a composition comprising anti-PD-1 antibodies or antigen binding fragments through a mixed mode AEX chromatographic process, comprising the steps:
(a) passing a load fluid comprising the composition through the mixed-mode anion exchange resin under a loading operating condition; and
(b) collecting the anti-PD-1 antibodies or antigen binding fragments in a flowthrough; wherein the anti-PD-1 antibodies or antigen binding fragments comprise: (a) light chain CDRs 1, 2 and 3 of SEQ ID NOs: 1, 2, and 3, respectively, and (b) heavy chain CDRs 1, 2 and 3 of SEQ ID NOs: 6, 7, and 8, respectively.

In one aspect, provided herein is a method of separating a host cell proteins from a composition comprising anti-PD-1 antibodies or antigen binding fragments and a host cell protein through a mixed mode AEX chromatographic process, comprising the steps:
(a) passing a load fluid comprising the composition through the mixed-mode anion exchange resin under a loading operating condition; and
(b) collecting the anti-PD-1 antibodies or antigen binding fragments in a flowthrough; wherein the anti-PD-1 antibodies or antigen binding fragments comprise: (a) light chain CDRs 1, 2 and 3 of SEQ ID NOs: 1, 2, and 3, respectively, and (b) heavy chain CDRs 1, 2 and 3 of SEQ ID NOs: 6, 7, and 8, respectively.

In another aspect, provided herein is a method of separating a host cell lipase from a composition comprising anti-PD-1 antibodies comprising main species, acidic species, and basic species, and a host cell lipase through a mixed-mode anion exchange (AEX) chromatographic process, comprising:
(a) passing a load fluid comprising the composition through the mixed-mode anion exchange resin under a loading operating condition with a conductivity of 2-50 mS/cm, while maintaining the load challenge of the anti-PD-1 antibodies from about 300 g/L to 400 g/L; and
(b) collecting the anti-PD-1 antibodies in a flowthrough;
   wherein the main species consist of two light chains and two heavy chains, wherein the two light chains consist of the amino acid sequence set forth in SEQ ID NO:5, wherein the two heavy chains consist of the amino acid sequence set forth in SEQ ID NO:11.

In another aspect, provided herein is a method of purifying anti-PD-1 antibody main species from a composition comprising anti-PD-1 antibodies comprising main species, acidic species, and basic species through a mixed-mode anion exchange (AEX) chromatographic process, comprising:
(a) passing a load fluid comprising the composition through the mixed-mode anion exchange resin under a loading operating condition with a conductivity of 2-50 mS/cm, while maintaining the load challenge of the anti-PD-1 antibodies from about 300 g/L to 400 g/L; and
(b) collecting the anti-PD-1 antibodies in a flowthrough;
   wherein the main species consist of two light chains and two heavy chains, wherein the two light chains consist of the amino acid sequence set forth in SEQ ID NO:5, wherein the two heavy chains consist of the amino acid sequence set forth in SEQ ID NO:11.

In one embodiment, about 50-80% main species is obtained in the flowthrough.

In another embodiment, the amount of acidic species in the flowthrough is about 5-25%, the amount of main species in the flowthrough is about 55-80%, the amount of basic species in the flowthrough is about 12-27%. In another embodiment, the basic species comprises basic 1 species, and less than about 15% of basic 1 species is obtained in the flowthrough. In one embodiment, the amount of basic 1 species in the flowthrough is about 4-12%.

In one embodiment, the amount of acidic species in the flowthrough is at about 5-25%. In one embodiment, the amount of acidic species in the flowthrough is about 1-10%. In one embodiment, the amount of acidic species in the flowthrough is about 6-10%. In one embodiment, the amount of acidic species in the flowthrough is at about 15.0-17.0%.

In one embodiment, the amount of main species in the flowthrough is about 50-85%. In another embodiment, the amount of main species in the flowthrough is at about 70-80%. In another embodiment, the amount of main species in the flowthrough is at about 70-85%. In one embodiment, the amount of main species in the flowthrough is about 55-65%.

In yet another embodiment, the amount of basic species in the flowthrough is about 12-27%. In yet another embodiment, the amount of basic species in the flowthrough is about 15-25%. In yet a further embodiment, the amount of basic 1 species in the flowthrough is about 4-12%. In yet a further embodiment, the amount of basic 1 species in the flowthrough is about 8-12%. In yet a further embodiment, the amount of basic 1 species in the flowthrough is about 5-9%. In one embodiment, the amount of basic 1 species is about 9-13% in the flowthrough.

In one embodiment, the amount of acidic species in the flowthrough is at about 5-25%, the amount of main species in the flowthrough is about 50-85%, the amount of basic species in the flowthrough is about 12-27%, and the amount of basic 1 species in the flowthrough is about 4-12%.

In another aspect, provided herein is a method of increasing the anti-PD-1 antibody main species in a composition comprising anti-PD-1 antibodies comprising main species, acidic species, and basic species through a mixed-mode anion exchange (AEX) chromatographic process, comprising:
(a) passing a load fluid comprising the composition through the mixed-mode anion exchange resin under a loading operating condition with a conductivity of 2-50 mS/cm, while maintaining the load challenge of the anti-PD-1 antibodies from about 300 g/L to 400 g/L; and
(b) collecting the anti-PD-1 antibodies in a flowthrough;
   wherein the main species consist of two light chains and two heavy chains, wherein the two light chains consist of the amino acid sequence set forth in SEQ ID NO:5, wherein the two heavy chains consist of the amino acid sequence set forth in SEQ ID NO:11.

In one embodiment, the amount of main species in the flowthrough increases by about 1-6% after the AEX mixed-mode chromatography. In one embodiment, the amount of main species in the flowthrough increases by about 2-5% after the AEX mixed-mode chromatography. In another embodiment, the amount of basic 1 species in the flowthrough decreases by about 1-4% after the AEX mixed-mode chromatography. In another embodiment, the amount of basic 1 species in the flowthrough decreases by about 2-4% after the AEX mixed-mode chromatography.

In the foregoing methods of the invention, in one embodiment, the loading operating condition has a conductivity of 2-50 mS/cm. In one embodiment, the loading operating condition has a conductivity of 2-25 mS/cm. In one embodiment, the loading operating condition has a conductivity of 2-20 mS/cm. In one embodiment, the loading operating condition has a conductivity of 4-20 mS/cm. In one embodiment, the loading operating condition has a conductivity of 10-15 mS/cm. In another embodiment, the loading operating condition has a conductivity of 8-15 mS/cm. In another embodiment, the loading operating condition has a conductivity of 8-14 mS/cm. In a further embodiment, the loading operating condition has a conductivity of 8-10 mS/cm.

In the foregoing methods of the invention, in one embodiment, the load challenge is up to 400 g/L of the anti-PD-1 antibodies or antigen binding fragments thereof. In one embodiment, the load challenge is about 300-400 g/L of anti-PD-1 antibodies or antigen binding fragments thereof. In one embodiment, the load challenge is about 200-400 g/L of anti-PD-1 antibodies or antigen binding fragments thereof. In another embodiment, the load challenge is about 400 g/L of anti-PD-1 antibodies or antigen binding fragments thereof. In yet another embodiment, step a) is paused if the load challenge is more than 400 g/L.

In the foregoing methods of the invention, in one embodiment, the loading operating condition has a pH of about 5.5 to about 7.5. In another embodiment, the loading operating condition has a pH of about 6.5 to about 7.5. In another embodiment, the loading operating condition has a pH of about 7.1 to about 7.3. In another embodiment, the loading operating condition has a pH of about 6.0 to about 7.5. In another embodiment, the loading operating condition has a pH of about 6.2 to about 7.2. In another embodiment, the loading operating condition has a pH of about 7.0 to about 7.4.

In the foregoing methods of the invention, in one embodiment, the loading operating condition has a conductivity of 8-14 mS/cm, and a pH of about 7.0 to 7.4. In another embodiment, the loading operating condition has a pH of about 6.0 to 7.5, and a conductivity of about 2-15 mS/cm. In another embodiment, the loading operating condition has a pH of about 6.2 to 7.2, and a conductivity of about 10-15 mS/cm.

In the foregoing methods of the invention, in one embodiment, the salt in the load fluid is selected from the group consisting of sodium chloride, sodium acetate, sodium phosphate, and Tris-HCl. In one embodiment, the salt is sodium chloride. In another embodiment, the salt is sodium acetate. In yet another embodiment, the salt is sodium phosphate. In another embodiment, the salt is sodium chloride and sodium phosphate. In one embodiment, the concentration of sodium chloride in the load fluid is about 5 mM, the concentration of sodium phosphate in the load fluid is about 25 mM, and the pH of the load fluid is from about 7.1 to about 7.3.

In the foregoing methods of the invention, in one embodiment, the AEX mixed-mode chromatography resin provides ligands that interact with the host cell lipase, anti-PD-1 antibodies or antigen binding fragments thereof, or a combination thereof through hydrogen bonding, thiophilic, hydrophilic, and/or hydrophobic, and ionic intermolecular interactions. In other embodiments, the AEX mixed-mode chromatography resin provides ligands that interact with the host cell lipase, anti-PD-1 antibodies or antigen binding fragments thereof, or a combination thereof through hydrogen bonding, thiophilic, and/or hydrophobic, and ionic intermolecular interactions. In other embodiments, the AEX mixed-mode chromatography resin provides ligands that interact with the host cell lipase, anti-PD-1 antibodies or antigen binding fragments thereof, or a combination thereof through hydrophobic and ionic intermolecular interactions. In a preferred embodiment, the mixed-mode AEX comprises a positively charged aromatic compound. In a preferred embodiment, the mixed-mode AEX is Capto Adhere (from Cytiva). In another preferred embodiment, the mixed-mode AEX resin comprises N-benzyl-n-methyl ethanolamine attached to the resin. In a preferred embodiment, the mixed-mode AEX is Nuvia aPrime 4A (from BioRad). In another preferred embodiment, the mixed-mode AEX resin comprises

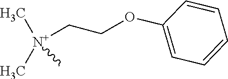

attached to the resin.

The methods of separation provided herein can be used in combination with one or more separation steps described herein or commonly used in the art. In one embodiment, one or more separation steps precede the method described herein. In another embodiment, one or more separation steps follow the method described herein. In yet another embodiment, one or more separation steps are performed between two methods described herein. In still other embodiments, one or more separation steps are performed before, after, and/or between the methods described herein. There is no limitation of how many separation steps or methods can be combined or the order of the separation steps or methods to be combined.

In more embodiments of the various methods provided herein, the load fluid is an eluate from a prior chromatographic process. In one embodiment, the prior chromatographic process comprises an affinity chromatography. In another embodiment, the prior chromatographic process comprises an affinity chromatography followed by an ion exchange chromatography. In yet another embodiment, the affinity chromatography is a protein A chromatography. In still another embodiment, the ion exchange chromatography is an AEX chromatography. In yet still another embodiment, the prior chromatographic process comprises a protein A chromatography followed by an AEX chromatography. In one embodiment, the load fluid is an eluate from a protein A chromatography performed in bind and elute mode followed by AEX chromatography performed in flowthrough mode.

### IV. Host Cell Proteins (HCP)

The methods provided in Section III apply to a broad variety of HCP. The HCP can be any endogenous protein derived from a host cell (e.g., CHO cell) during bioprocessing of anti-PD-1 antibodies or antigen binding fragments expressed in the host cell. Non-limiting examples of HCP include structural protein, functional protein, secreted protein, enzyme, such as lipase, proteinase, and kinase, etc. In some embodiments, the HCP is a structural protein. In certain embodiments, the HCP is a functional protein. In other embodiments, the HCP is a secreted protein. In yet another embodiment, the HCP is an enzyme. In one embodiment, the HCP is a lipase. In another embodiment, the HCP is a proteinase. In yet another embodiment, the HCP is a kinase. In one embodiment, the HCP is Clusterin.

In certain embodiments, the lipase is selected from the group consisting of PLBL2, LPL, LPLA2, LP-PLA2, and LAL. In one embodiment, the lipase is PLBL2. In another embodiment, the lipase is LPL. In yet another embodiment, the lipase is LPLA2. In one embodiment, the lipase is LP-PLA2. In another embodiment, the lipase is LAL. In still another embodiment, the lipase includes two, three, four, five, six, seven, eight, nine, ten, or more different lipases. In yet still another embodiment, the lipase includes two, three, four, or five different lipases selected from the group consisting of PLBL2, LPL, LPLA2, LP-PLA2, and LAL. In one embodiment, the lipase includes PLBL2 and LPL. In another embodiment, the lipase includes PLBL2 and LPLA2. In yet another embodiment, the lipase includes PLBL2 and LP-PLA2. In still another embodiment, the lipase includes PLBL2 and LAL. In one embodiment, the lipase includes LPL and LPLA2. In another embodiment, the lipase includes LPL and LP-PLA2. In yet another embodiment, the lipase includes LPL and LAL. In still another embodiment, the lipase includes LPLA2 and LP-PLA2. In one embodiment, the lipase includes LPLA2 and LAL. In another embodiment, the lipase includes LP-PLA2 and LAL. In yet another embodiment, the lipase includes PLBL2, LPL, and LPLA2. In still another embodiment, the lipase includes PLBL2, LPL, and LP-PLA2. In one embodiment, the lipase includes PLBL2, LPL, and LAL. In another embodiment, the lipase includes PLBL2, LPLA2, and LP-PLA2. In yet another embodiment, the lipase includes PLBL2, LPLA2, and LAL. In still another embodiment, the lipase includes PLBL2, LP-PLA2, and LAL. In one embodiment, the lipase includes LPL, LPLA2, and LP-PLA2. In another embodiment, the lipase includes LPL, LPLA2, and LAL. In yet another embodiment, the lipase includes LPL, LP-PLA2, and LAL. In still another embodiment, the lipase includes LPLA2, LP-PLA2, and LAL. In one embodiment, the lipase includes PLBL2, LPL, LPLA2, and LP-PLA2. In another embodiment, the lipase includes PLBL2, LPL, LPLA2, and LAL. In yet another embodiment, the lipase includes PLBL2, LPL, LP-PLA2, and LAL. In still another embodiment, the lipase includes PLBL2, LPLA2, LP-PLA2, and LAL. In yet still another embodiment, the lipase includes PLBL2, LPL, LPLA2, LP-PLA2, and LAL.

The host cell can be any cell used for expressing an exogenous protein. Common host cells used in manufacturing of biopharmaceuticals include but are not limited to CHO cell, baby hamster kidney (BHK21) cell, murine myeloma NS0 cell, murine myeloma Sp2/0 cell, human embryonic kidney 293 (HEK293) cell, fibrosarcoma HT-1080 cell, PER.C6 cell, HKB-11 cell, CAP cell, HuH-7 cell, murine C127 cell, and a naturally generated or genetically modified variant thereof. In certain embodiments, the host cell is CHO cell. In some embodiments, the host cell is baby hamster kidney (BHK21) cell. In other embodiments, the host cell is murine myeloma NS0 cell. In yet other embodiments, the host cell is murine myeloma Sp2/0 cell. In still other embodiments, the host cell is human embryonic kidney 293 (HEK293) cell. In certain embodiments, the host cell is fibrosarcoma HT-1080 cell. In some embodiments, the host cell is PER.C6 cell. In other embodiments, the host cell is HKB-11 cell. In yet other embodiments, the host cell is CAP cell. In still other embodiments, the host cell is HuH-7 cell. In certain embodiments, the host cell is murine C127 cell. In some embodiments, the host cell is a naturally generated variant of the above host cell. In other embodiments, the host cell is a genetically modified variant of the above host cell.

In certain embodiments, the CHO cell lipase is selected from the group consisting of PLBL2, LPL, LPLA2, LP-PLA2, and LAL. In one embodiment, the CHO cell lipase is PLBL2. In another embodiment, the CHO cell lipase is LPL. In yet another embodiment, the CHO cell lipase is LPLA2. In one embodiment, the CHO cell lipase is LP-PLA2. In another embodiment, the CHO cell lipase is LAL. In still another embodiment, the CHO cell lipase includes two, three, four, five, six, seven, eight, nine, ten, or more different CHO cell lipases. In yet still another embodiment, the CHO cell lipase includes two, three, four, or five different CHO cell lipases selected from the group consisting of PLBL2, LPL, LPLA2, LP-PLA2, and LAL. In one embodiment, the CHO cell lipase includes PLBL2 and LPL. In another embodiment, the CHO cell lipase includes PLBL2 and LPLA2. In yet another embodiment, the CHO cell lipase includes PLBL2 and LP-PLA2. In still another embodiment, the CHO cell lipase includes PLBL2 and LAL. In one embodiment, the CHO cell lipase includes LPL and LPLA2. In another embodiment, the CHO cell lipase includes LPL and LP-PLA2. In yet another embodiment, the CHO cell lipase includes LPL and LAL. In still another embodiment, the CHO cell lipase includes LPLA2 and LP-PLA2. In one embodiment, the CHO cell lipase includes LPLA2 and LAL. In another embodiment, the CHO cell lipase includes LP-PLA2 and LAL. In yet another embodiment, the CHO cell lipase includes PLBL2, LPL, and LPLA2. In still another embodiment, the CHO cell lipase includes PLBL2, LPL, and LP-PLA2. In one embodiment, the CHO cell lipase includes PLBL2, LPL, and LAL. In another embodiment, the CHO cell lipase includes PLBL2, LPLA2, and LP-PLA2. In yet another embodiment, the CHO cell lipase includes PLBL2, LPLA2, and LAL. In still another embodiment, the CHO cell lipase includes PLBL2, LP-PLA2, and LAL. In one embodiment, the CHO cell lipase includes LPL, LPLA2, and LP-PLA2. In another embodiment, the CHO cell lipase includes LPL, LPLA2, and LAL. In yet another embodiment, the CHO cell lipase includes LPL, LP-PLA2, and LAL. In still another embodiment, the CHO cell lipase includes LPLA2, LP-PLA2, and LAL. In one embodiment, the CHO cell lipase includes PLBL2, LPL, LPLA2, and LP-PLA2. In another embodiment, the CHO cell lipase includes PLBL2, LPL, LPLA2, and LAL. In yet another embodiment, the CHO cell lipase includes PLBL2, LPL, LP-PLA2, and LAL. In still another embodiment, the CHO cell lipase includes PLBL2, LPLA2, LP-PLA2, and LAL. In yet still another embodiment, the CHO cell lipase includes PLBL2, LPL, LPLA2, LP-PLA2, and LAL.

### Compositions of Anti-PD-1 Antibodies or Antigen-Binding Fragments.

The present disclosure also provides a composition of anti-human PD-1 antibodies or antigen-binding fragments obtainable by or produced by the methods disclosed herein.

### EXAMPLES

### Example 1: Resin Screens for PLBL2 Clearance in Flowthrough Mode

### Screening Methodology

To screen resins for PLBL2 removal, a tiered high throughput approach was utilized. Partition coefficients (Kp) for each species from slurry plate experiments were used to calculate selectivity coefficients (α) to predict resolution between pembrolizumab and PLBL2 at a range of mobile phase conditions on ion exchange (IEX) and multimodal chromatography resins. This data was used to inform pH/conductivity conditions evaluated using robocolumns. Resins were evaluated based on capability of clearing PLBL2 when operated in flowthrough mode. Yield and HMW clearance were also monitored in addition to PLBL2 levels.

### Cation Ion Echange (CEX): Poros HS50

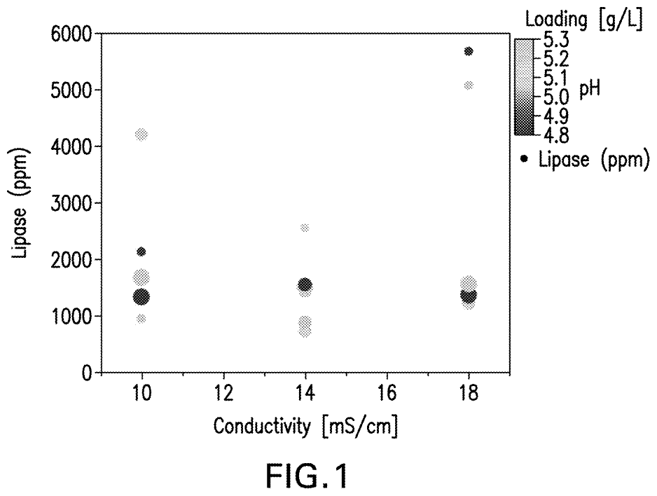

The cation exchanger Poros HS50 was evaluated for PLBL2 clearance in flow through mode at mobile phase conditions of pH 5.8-5.3, conductivities of 10-20 mS/cm up to 200 g/L loading. Despite clearance capabilities demonstrated for residual impurities and High Molecular Weight (HMW) species, flow through operation of POROS 50 HS did not remove PLBL2. In fact, at most conditions evaluated, PLBL2 levels in the CEX pool (CEXP) were higher than load levels. One hypothesis is that mAb binding to the resin was stronger than that of PLBL2 at these conditions, leading to artificially inflated PLBL2 ppm levels in the CEXP. Another hypothesis is that the loading conditions used for the CEX step dissociates PLBL2 from the antibody and is hence more abundant in free solution. FIG. 1 shows that PLBL2 levels in the CEXP were a function of pH, conductivity, and loading. The effect of conductivity was convex, with a minimum PLBL2 content at 14 mS/cm. At this optimal conductivity, PLBL2 levels were lower at the higher load (125-200 g/L) and pH>5.1 conditions. The lowest PLBL2 levels were observed at the design center point of pH 5.0, 14 mS/cm, 125 g/L load, at which conditions the PLBL2 levels were equivalent to those in the load (~800 ppm). Since no PLBL2 clearance was observed Poros HS 50 was not pursued further.

### Multimodal Cation Exchange (MMCEX): Capto MMC

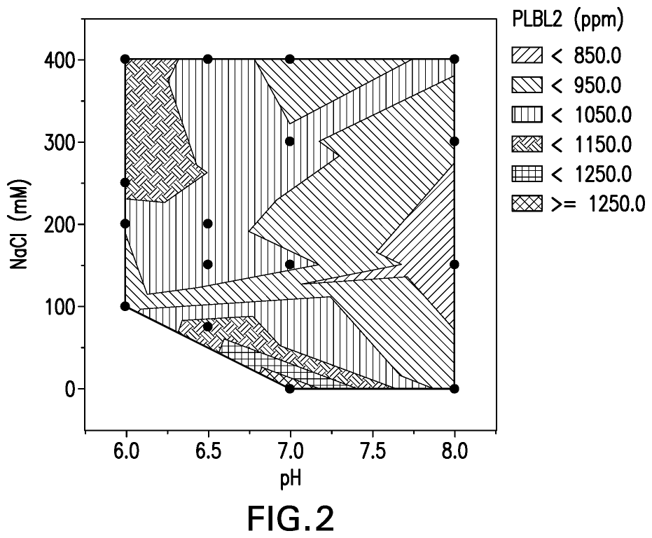

The multimodal cation exchanger Capto MMC was evaluated for PLBL2 clearance in flowthrough mode at mobile phase conditions of pH 6-8, 0-400 mM NaCl up to 100 g/L loading. Capto MMC did not provide clearance of PLBL2 across the range of conditions evaluated, similar to what was observed on POROS HS 50. FIG. 2 shows PLBL2 levels in the MMCEXP. The lowest PLBL2 levels were observed at pH 8, 150 mM NaCl. Even at this condition, pool PLBL2 levels were close to that of the feed (~800 ppm). Based on the outcome of the PLBL2 readout, MMCEX was not pursued further.

### Mixed Mode Anion Ion Exchange Chromatography (MMAEX): Capto Adhere

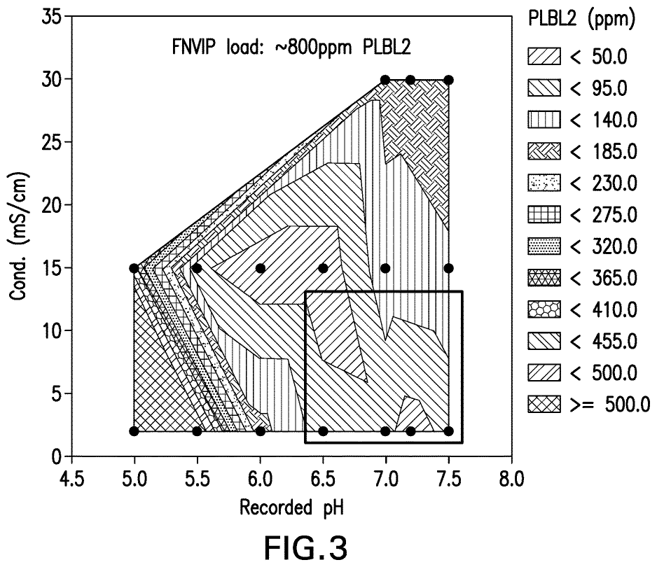

The multimodal anion exchanger Capto Adhere was evaluated for PLBL2 clearance in flow through mode at mobile phase conditions ranging from pH 5-7.5, conductivity 2-30 mS/cm for loadings up to 300 g/L. These screening conditions were determined based on the molecular properties of pembrolizumab and PLBL2 and included pH conditions above and below pembrolizumab pI. FIG. 3 summarizes the PLBL2 clearance for the Capto Adhere screen. PLBL2 clearance was non-linearly related to pH and conductivity. The lowest PLBL2 levels were observed at moderate pH/conductivity conditions (~pH 5.5-6.5, 15 mS/cm) or at high pH/low conductivity (pH 6.5-7.5, 15 mS/cm). As shown in FIG. 3, the Filtered Neutralized Viral Inactivation Product (FNVIP) feed had approximately 800 ppm of PLBL2 and the best performing conditions resulted in pool levels <150 ppm. Moderate yields between 65-75% were observed and HMW levels were <0.25% at all conditions evaluated. The initial screens indicated potential for PLBL2 clearance with good HMW clearance with potential process robustness benefits. As indicated in FIG. 3, we identified a region of interest between pH 6.5-7.5, 2-10 mS/cm as an area to further evaluate.

### Example 2: Optimization of Capto Adhere MM AEX Operating Conditions for PLBL2 Clearance

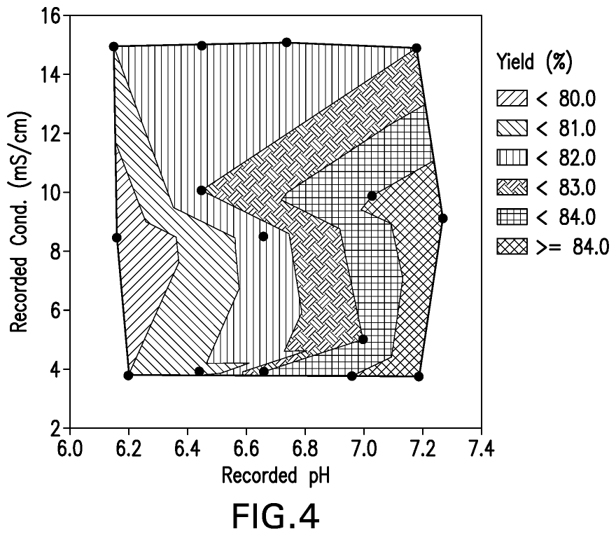

JMP statistical analysis software (from SAS) was used to develop partial least squares (PLS) models based on the experimental results from the initial Capto Adhere screen. These models were then used as input to Monte Carlo simulations which were used to narrow the optimization range to pH 6.2-7.2, 4-15 mS/cm as described below. For each pH/conductivity condition, the column load was 400 g/L and flow through fractions were collected in 100 g/L increments for analytical analysis. Product samples were analyzed for concentration (yield), HMW content and PLBL2 levels. Loading was increased from 200-300 g/L in the initial screen to 400 g/L in the optimization screen to achieve higher yields. FIG. 4 indicates that yields above 79% were observed across the region of interest. pH was directly related to yield and had a greater impact at low conductivities. The conductivity dependence became non-linear above pH 6.5. The overall yield trends were subtle, varying within 5%.

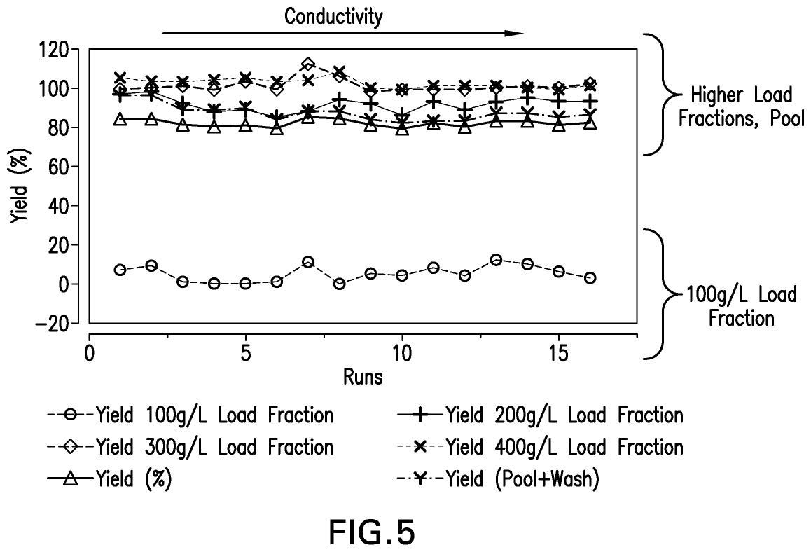

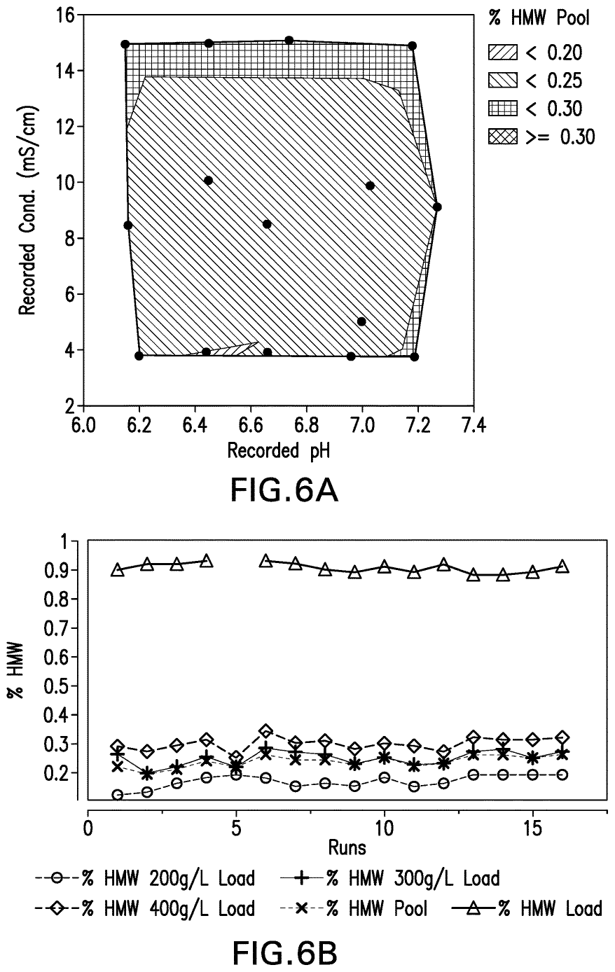

FIG. 5 breaks down the yield data by product fraction. Minimal recovery was observed at all conditions at the 100 g/L load, indicating some interaction between pembrolizumab and the Capto Adhere resin. This pH range straddles the pembrolizumab pI (6.8-6.9) so the mAb net charge transitions from weakly positively charged to weakly negatively charged. Product fractions were also analyzed for HMW content using the platform Ultra Performance Size Exclusion Chromatography Arginine (UP-SEC Arg) method. FIG. 6 summarizes HMW clearance over Capto Adhere for the product pool (6A) and the product fractions (6B). As can be seen in the FIG. 6, Capto Adhere provides robust HMW clearance across the full range of conditions evaluated. HMW levels were reduced from 0.9% in the FNVIP load to <0.26% in the MM AEXP. Furthermore, HMW clearance did not exhibit a strong load dependence up to 400 g/L.

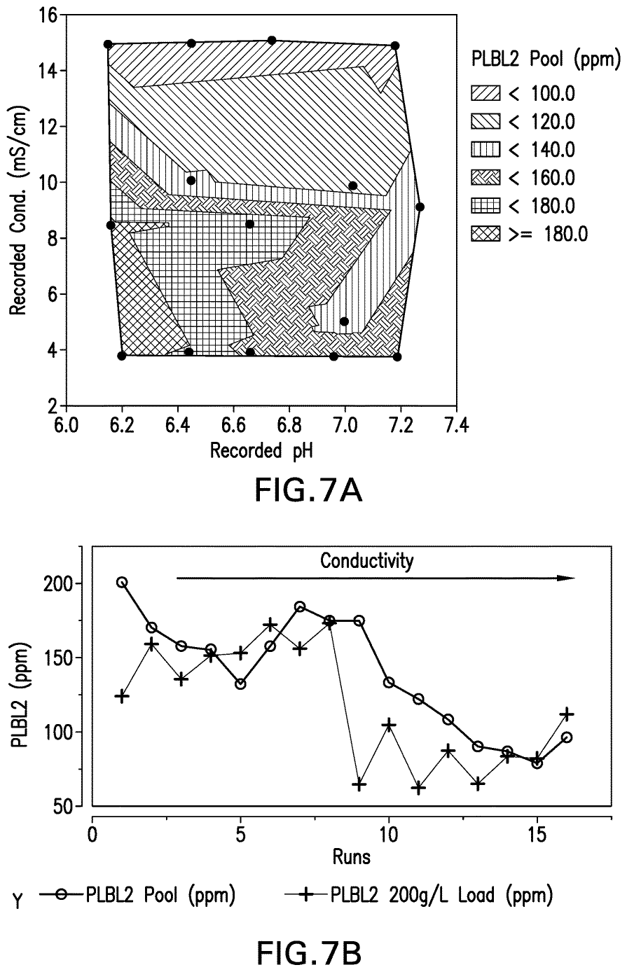

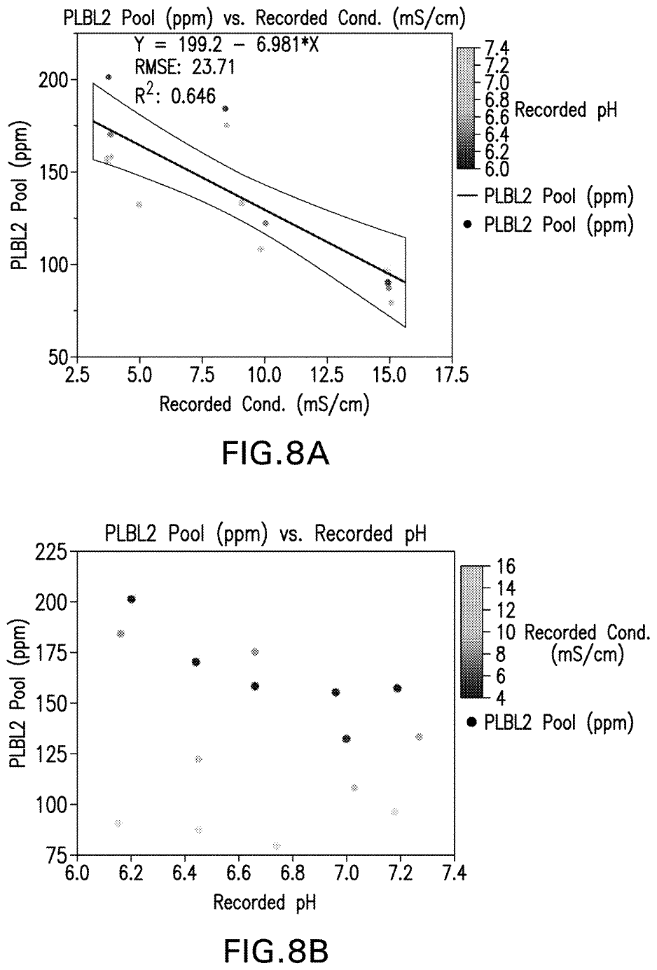

Importantly, Capto Adhere also provided PLBL2 clearance at these conditions, particularly at moderate conductivities (8-15 mS/cm). FIG. 7 summarizes the PLBL2 levels in the product pool and in the 200 g/L load fraction. In the mock pool (FIG. 7A), PLBL2 levels were inversely related to pH and conductivity. The pH dependence weakened as conductivity increased. The lowest PLBL2 levels were observed at 15 mS/cm, pH 6.2-7.2. Loading did impact PLBL2 clearance, particularly at the higher conductivities, where the best PLBL2 clearance was observed (FIG. 7B). As shown in FIG. 8A, PLBL2 clearance showed a strong correlation with conductivity in this range (R²-0.65) and was not correlated with pH (FIG. 8B).

Based on this data, we identified potential operating conditions of pH 7.0±0.2 and 14±1 mS/cm for flow through removal of PLBL2 on Capto Adhere.

### Example 3: Operating Considerations of Capto Adhere MMAEX Chromatography for Integration into Continuous Process

Beyond the flow through constraint, another important consideration during polishing step development in the continuous pembrolizumab context was integration with the surrounding drug substance process steps. To eliminate a pH adjustment between the polishing chromatography steps, it was desired to shift the MM AEX pH range from 7.0±0.2 to 7.2±0.2 to align with the AEX specification. To support this shift and to more fully understand the impact of conductivity in this pH range, an additional round of screening was conducted at pH 6.8-7.4, conductivities 12-20 mS/cm.

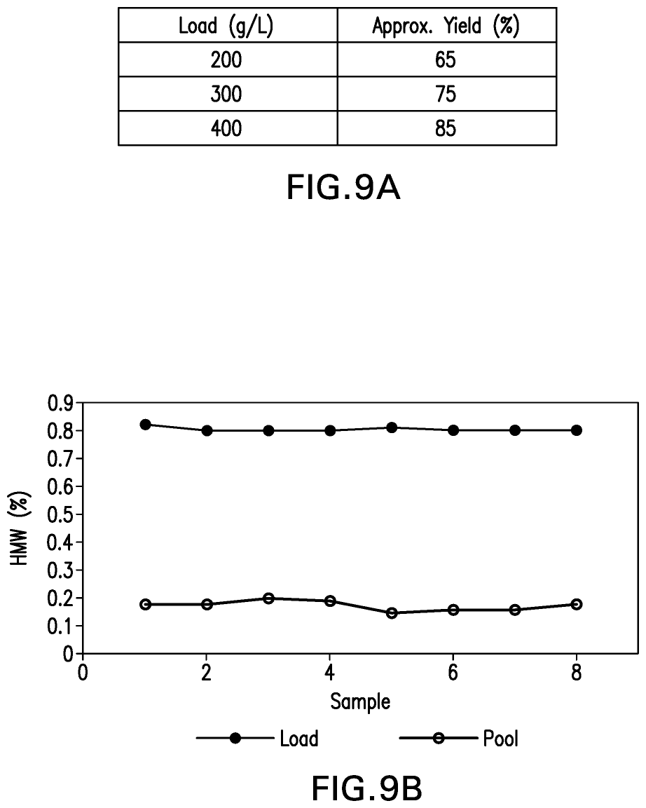

FIG. 9 summarizes the yield and HMW results for the pH/conductivity optimization screen. FIG. 9A demonstrates that yield increases as a function of loading. Similar yield trends were observed across the pH/conductivity space, with a slight increase in yield observed at 20 mS/cm vs. 12 ms/cm conductivity. pH had minimal impact on yield in the 6.8-7.4 range. Based on this data, the load criteria is 300-400 g/L (350 g/L target). FIG. 9B illustrates the robust HMW clearance obtained on Capto Adhere. HMW levels were reduced from >0.80% to <0.2% and clearance was independent of pH or conductivity between pH 6.8-7.4, up to 20 mS/cm conductivity.

### Scale Up Evaluation

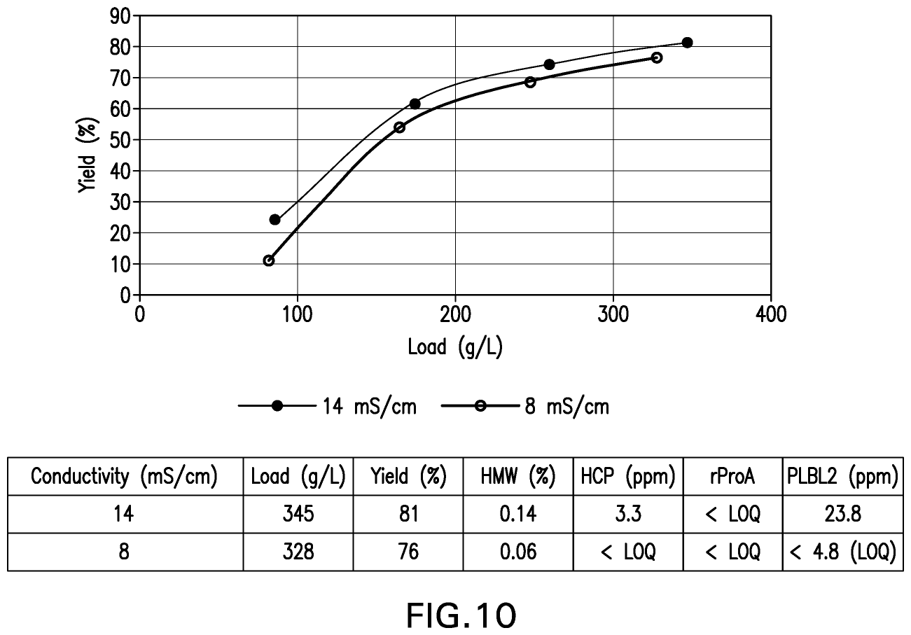

Bench scale runs using a 4 mL Adhere column were conducted at the centerpoint loading of 350 g/L and high (14 mS/cm) and low (8 mS/cm) conductivities. The results are summarized in FIG. 10. Although sharp, product breakthrough is delayed and is a function of conductivity, occurring between 7-12 CV for conductivities 8-14 mS/cm. This contributes to low yield observed through the first 100 g/L loading (FIG. 10). The product quality data in FIG. 10 shows that both high and low conductivity conditions provided sufficient HMW, residuals, and PLBL2 clearance. Based on these results, the conductivity specification was set to 8-10 mS/cm with a product collection criteria of 10CV after the start of loading.

### Example 4: Scale up Conditions and Control

For scale up operations, the Capto Adhere polishing step is operated according to the conditions outlined in Table 3. This step is operated in flow-through mode with a target load of 400 g/L of pembrolizumab on mixed-mode anion exchanger Capto Adhere (Cytiva). Residual impurities, particularly PLBL2 and HMW species bind to the resin while the product flows through. Mixed-mode AEX Load is prepared by using 4M NaCl to increase the conductivity of AEX Pool to a target of 9 mS/cm (+0.5 mS/cm deadband) followed by 0.22 μm filtration. The column is equilibrated and washed using a 25 mM NaPhosphate, 5 mM NaCl, pH 7.2, 8.4 mS/cm buffer. Product collection criteria is established based on a fixed Column Volume (CV) at a flowrate of 170 cm/hr and column effluent absorbance is monitored at 280 nm throughout.

TABLE 3
Operating conditions for Capto Adhere in pembrolizumab process

| pH (—) | Conductivity (mS/cm) | Load (g/L) |
|---|---|---|
| 7.2 ± 0.1 | 9 ± 1 | 400 |

A dynamic UV-based approach is used to control loading over the Capto Adhere step when operated as part of the integrated process train. In the absence of dynamic control, the loading is determined by defining a target volume. This means that the amount loaded can change as the concentration of the load fluid varies, for example, during the non-steady-state operations of start up and shut down. Dynamic control utilizes an in-line UV sensor (optek-Danulat, Essen, Germany) to measure the concentration to the Capto Adhere column in real time and thus provides the capability to account for changes in the load fluid concentration. The absorbance curve from the UV sensor is integrated instantaneously to determine column loading. The real-time calculated column load challenge value then is used to determine the process command (i.e. continue loading or complete load and proceed to wash) via the automation control software Delta V.

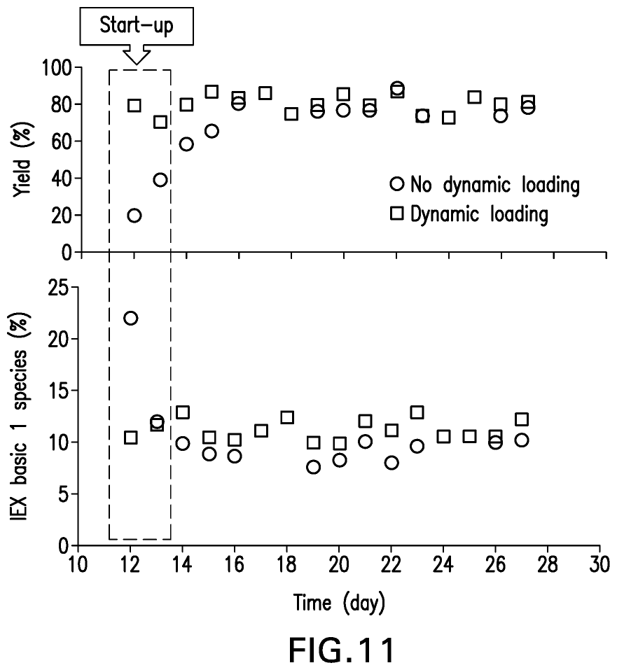

An example of the impact of using dynamic loading is given in FIG. 11 for the flow through MMAEX (Capto Adhere) step compared to the non-dynamic version based on fixed volume feed. When the column load challenge reaches 400 g/L, the loading phase is paused, and the column goes through a washing phase, and sanitization prior to starting the next round of the loading phase. From development data, low mass column loadings on the MMAEX step (<300 g/L) lead to shifts in the charge variant profile (increased basic 1 peak in the charge variant profile; data not shown) and reduced step yield (FIG. 5). This effect is clearly seen in FIG. 11, where during the start-up phase for the static, fixed volume method, the initial feed material at lower concentration results in a lower yield and increased basic 1 species. When utilizing the dynamic loading methodology to control the mass loading of the column both yield and charge variant profile are stable.

### Example 5: PLBL2 ELISA Assay

A PLBL2 ELISA was used for quantitation of residual PLBL2 in pembrolizumab. This sandwich ELISA utilizes several custom reagents. Recombinant PLBL2 was custom generated in a CHO-E37 cell line and purified by GenScript (Piscataway, NJ). This PLBL2 standard was used to inoculate rabbits at SDIX (Newark, DE) for generation of purified anti-PLBL2 polyclonal antibodies, referred to as capture antibody. A portion of the capture antibody was conjugated with biotin using an EZ-LINK Sulfo-NHS-LC-Biotinylation kit purchased from ThermoScientific (Waltham, MA). This biotinylated rabbit anti-PLBL2 polyclonal antibody is referred to as detection antibody. A summary of these custom reagents and other commercially available reagents used are provided in Table 4.

The PLBL2 ELISA was carried out at room temperature using the following procedure, summarized in Table 5. For all instances, wash buffer was 1×PBS, 0.05% Tween-20 and assay diluent was 90:10 wash buffer: 1% casein in PBS. Between each step the plate was washed, 300 μL wash buffer per well, four times total then patted dry. Plates were coated using 110 μL per well of capture antibody in 1×PBS (2.0 g/mL) for 120±10 minutes, shaken at 450 RPM. 275 μL of 1% casein in PBS was added to each well to block the plate for 60-120 minutes, shaken at 450 RPM. 100 μL of standards and samples diluted in assay diluent were added to each well for 60±5 minutes, shaken at 450 RPM. 100 μL of detection antibody in assay diluent (2.0 μg/mL) was added to each well for 60±5 minutes, shaken at 450 RPM. 100 μL of NeutrAvidin-horseradish peroxidase (HRP) diluted 3000-fold in assay diluent was added to each well for 30±5 minutes, shaken at 450 RPM. 100 μL of LumiGLO substrate was added to each well. After this addition, the plate was covered from light and allowed to sit for 10±5 minutes. After the wait time, the plate was read on a SpectraMax M5e. For chemiluminescence the critical SpectraMax M5e settings used were: read mode (endpoint), luminescence LM1 (all), PMT and optics integration (500 ms), number of reads (1), calibrate (on), temperature (ambient). A standard curve was generated using mean values of the standards and the 4-parameter curve fit function.

TABLE 4
List of Reagents Used in Assay

| Reagent Name | Vendor | Catalog Number |
|---|---|---|
| Capture Antibody | Custom (SDIX) | N/A |
| Detection Antibody | Custom (SDIX) | N/A |
| PLBL2 Standard | Custom (GenScript) | N/A |
| NeutrAvidin-HRP | ThermoScientific | 31001 |
| LumiGLO | SeraCare | 5430-0040 |

TABLE 5
PLBL2 ELISA Procedure

| Step | Contents | Volume/Well | Incubation Time |
|---|---|---|---|
| Plate coated with | 2.0 μg/mL capture | 110 μL | 120 ± 10 min OR |
| capture antibody | antibody in 1x PBS | | 12-24 hr at 4° C. |
| Wash step | Wash buffer | 300 μL (4x) | N/A |
| Plate Blocking | 1% (w/v) Casein in PBS | 275 μL | 60-120 min OR |
| with Casein | | | 12-24 hr at 4° C. |
| Wash step | Wash buffer | 300 μL (4x) | N/A |
| Incubation of | Various dilutions in | 100 μL | 60 ± 5 min |
| samples and | assay diluent | | |
| standards | | | |
| Wash step | Wash buffer | 300 μL (4x) | N/A |
| Incubation of | 2.0 μg/mL detection | 100 μL | 60 ± 5 min |
| detection | antibody in assay | | |
| antibody | diluent | | |
| Wash step | Wash buffer | 300 μL (4x) | N/A |
| Incubation of | 3000-fold dilution in | 100 μL | 30 ± 5 min |
| NeutrAvidin-HRP | assay diluent | | |
| Wash step | Wash buffer | 300 μL (4x) | N/A |
| Incubation of | 1:1 ratio of substrate | 100 μL | 10 ± 5 min |
| LumiGLO | A and substrate B | | |

### Example 6: Ion Exchange (IEX) Method to Measure Acidic Species of Anti-PD-1 Antibodies

For the IEX method, using a Waters Alliance LC system (Milford, MA, U.S.A.), the Thermo Scientific's ProPac WCX-10 (p/n: 054993, particle size 10 μm, diameter 4 mm, length 250 mm) was chosen with a loading of 80 μg sample. Mobile Phase (A) 24 mM MES pH 6.1 with 4% acetonitrile, and mobile phase (B) 20 mM sodium phosphate, 95 mM NaCl pH 8.0 with 4% acetonitrile was used as a non-linear, sigmoidal shape, pH gradient, and the separation was monitored over 34 min with a flow rate of 0.5 mL min⁻¹, with the column temperature being 35° C. The gradient used was: 22%-22% B for 0-0.6 min; 22%-29% B for 0.6-15.0 min; 29%-70% B for 15.0-30.0 min; 70%-100% B for 30.0-30.5 min; and 100%-100% B from 30.5-33.0 min. Mobile phase (C) 10 mM CHES pH 8.0, 40 mM Tris, 15 mM EDTA, 200 mM NaCl, and 4% acetonitrile was used to strip the column at 0.5 mL min⁻¹ from 33.1-34.0 min, followed by re-equilibration with 22% B from 34.5-44.5 min at 1.0 mL min⁻¹. From 44.5-45 min, the flowrate was reduced to 0.5 mL min⁻¹. The elution was monitored at 280 nm for the detection of peaks. The assay variability was determined to be within 1%.

The chemical composition of each identified peak of the pembrolizumab sample was determined by collecting samples of each peak, and performing peptide mapping and reverse phase liquid chromatography followed by mass-spectrometry according to the following methods with analysis performed by MS/MS.

### Sample Preparation

In-process or Drug Substance (DS) samples were diluted with water to 5 mg/mL. A total of 20 μL of the diluted sample (contains 100 μg) was denatured and reduced in final solution (100 μL) containing 6 M Guanidine-HCl, 50 μM Tris-HCl, 50 μM EDTA and 200 μM DTT. The mixed sample was incubated in thermomixer at 37° C. with 300 rpm shaking for 30 min. After mix and spin down, each sample is alkylated with 5 μL of odoacetamide (IAM) (1 M) protected from light at 25° C. for 30 min. A total of 5 μL of DTT (200 μM) was added to block the unreacted iodoacetamide (IAM). Lysyl endopeptidase (Lys-C) (Wako, 125-05061) enzyme (1:10 (wt:wt)) in 500 μL was added to the protein sample with slowly pipette up/down 3 times to mix well. The digestion was incubated in a thermomixer at 37° C. for 60 min. The digestion is quenched with 15 μL of 20% TFA. Digested sample was analyzed by LC-MS within 24 h after sample digestion. Otherwise, digested samples were stored at −80° C. for future analysis.

### LC-MS Methods and Data Analysis

Waters Acquity Liquid Chromatography was used to inject 20 μL of sample on the column (UPLC HSS T3 100 Å, 1.8 μm, 2.1 mm×150 mm, P/N: 186003540). Autosampler was set at 5° C. Mobile phase A was 0.02% TFA in water and mobile phase B was 0.02% TFA in acetonitrile with a gradient of 0.1% B from 0 to 5 min, 0.1%-10% B from 5 to 7 min, followed by a linear increase to 35% B over the next 38 min. Q Exactive Orbitrap MS (Thermo) was used to collect the MS1 data. Chromeleon and Xcalibur was used for data analysis.

The main peak was determined to predominantly contain the antibody with the amino acid sequence set forth in SEQ ID NO:5 and SEQ ID NO:11 for both light chains and heavy chains, respectively. Oxidation (for example, Methionine 105) and deamidation of asparagine residues (for example, N31, N52, N55, N59 or N61 in the heavy chain of SEQ ID NO: 11) of the foregoing antibody was detected in the Acidic Variants peak. Deamidation of asparagine residues (for example, N384, N389 or N390 in the heavy chain of SEQ ID NO: 11) of the foregoing antibody was detected in the Acidic1 peak. In the Basic 1 peak, antibodies with one heavy chain consisting of the amino acid sequence of SEQ ID NO: 11, one heavy chain consisting of the amino acid sequence of SEQ ID NO: 12, and two light chains consisting of the amino acid sequence of SEQ ID NO: 5; or one heavy chain consisting of the amino acid sequence of SEQ ID NO: 11, one heavy chain consisting of the amino acid sequence of SEQ ID NO: 14, wherein the C-terminal leucine is alpha-amidated, and two light chains consisting of the amino acid sequence of SEQ ID NO: 5 were detected.

The ion exchange chromatograms and % acidic species, main peak, and basic species from samples after Protein A chromatography (PAP), Anion exchange chromatography (AEXP) and mixed-mode AEX chromatography (MMAEX) using the dynamic control method (load challenge 300-400 g/L) produced from a continuous perfusion process (see WO2022165001) are illustrated in FIG. 12 and Table 6. The sum of the acidic species (acidic variants, Acidic 1 and Pre-Main) for the PAP, AEXP and MMAEX samples are 7.86%, 8.76%, and 10.8% respectively. The sum of the basic species (basic 1, basic variant A, basic2, basic variant B) for the PAP, AEXP and MMAEX samples are 16.95%, 18.19%, and 14.06% respectively. In other batches of pembrolizumab samples prepared by the continuous perfusion process, the main peak is about 74-80%.

The ion exchange chromatograms and % acidic species, main peak, and basic species from pembrolizumab obtained by fed-batch method are summarized in Table 7. The sum of the acidic species (acidic variants, Acidic 1 and Pre-Main) for the reference sample is 16.56%. The sum of the basic species (basic 1, basic variant A, basic2, basic variant B) for the reference sample is 23.76%. The sum of the basic variants (basic variant A and basic variant B) for the reference sample is 6.23%.

TABLE 6
Pembrolizumab obtained from the continuous perfusion
process: acidic, main and basic species

| | | | | | | | Basic |
|---|---|---|---|---|---|---|---|
| | | | | | | | Variants |
| Sample | Acidic | Acidic | Pre | | Basic | Basic | (Sum |
| Name | Variants | 1 | Main | Main | 1 | 2 | A + B) |
| PAP-D22 | 3.12 | 2.67 | 2.07 | 75.2 | 9 | 2.55 | 5.4 |
| AEXP-D22 | 3.77 | 3 | 1.99 | 73.05 | 9.96 | 2.72 | 5.51 |
| MMAEXP- | 3.41 | 2.19 | 2.60 | 77.74 | 6.52 | 3.16 | 4.38 |
| D22 | | | | | | | |

TABLE 7
Pembrolizumab from fed-batch process
acidic, main and basic species

| | Retention | |
|---|---|---|
| Name | Time | % Area |
| Acidic Variants | 9.546 | 8.1 |
| Acidic 1 | 10.513 | 6.41 |
| Pre Main | 11.79 | 2.05 |
| Main | 13.269 | 59.68 |
| Basic 1 | 14.726 | 8.07 |
| Basic Variant A | 16.782 | 1.36 |
| Basic 2 | 17.743 | 9.46 |
| Basic Variant B | 21.795 | 4.87 |

As discussed in Example 4, using the dynamic control method in the AEX mixed-mode chromatography, the % main species of the pembrolizumab increased by about 4.7% mainly due to the decrease in the % basic 1 species by about 3.4%. Therefore, the % main species is expected to be maintained within the range of 50-80% after the AEX mixed-mode chromatography step depending on the upstream cell culture process.

1-4. (canceled)

5. A method of purifying anti-PD-1 antibody main species from a composition comprising anti-PD-1 antibodies comprising main species, acidic species, and basic species through a mixed-mode anion exchange (AEX) chromatographic process, comprising:
(a) passing a load fluid comprising the composition through the mixed-mode anion exchange resin under a loading operating condition with a conductivity of 2-50 mS/cm, while maintaining the load challenge of the anti-PD-1 antibodies from about 300 g/L to 400 g/L; and
(b) collecting the anti-PD-1 antibodies in a flowthrough;
wherein the main species consist of two light chains and two heavy chains, wherein the two light chains consist of the amino acid sequence set forth in SEQ ID NO:5, wherein the two heavy chains consist of the amino acid sequence set forth in SEQ ID NO:11.

6. The method of claim 5, wherein about 50-80% or about 50-85% main species is obtained in the flowthrough.

7. The method of claim 5, wherein the basic species comprises basic 1 species, and less than about 15% of basic 1 species is obtained in the flowthrough.

8. The method of claim 5, wherein the amount of acidic species in the flowthrough is about 5-25%, the amount of main species in the flowthrough is about 55-80%, the amount of basic species in the flowthrough is about 12-27%.

9. The method of claim 7, wherein the amount of basic 1 species in the flowthrough is about 4-12%.

10. A method of increasing the anti-PD-1 antibody main species in a composition comprising anti-PD-1 antibody main species, acidic species, and basic species through a mixed-mode anion exchange (AEX) chromatographic process, comprising:
(a) passing a load fluid comprising the composition through the mixed-mode anion exchange resin under a loading operating condition with a conductivity of 2-50 mS/cm, while maintaining the load challenge of the anti-PD-1 antibodies from about 300 g/L to 400 g/L; and
(b) collecting the anti-PD-1 antibodies in a flowthrough;
wherein the main species consist of two light chains and two heavy chains, wherein the two light chains consist of the amino acid sequence set forth in SEQ ID NO:5, wherein the two heavy chains consist of the amino acid sequence set forth in SEQ ID NO:11.

11. The method of claim 10, wherein the amount of main species in the flowthrough increases by about 1-6% after the AEX mixed-mode chromatography.

12. The method of claim 10, wherein the amount of basic 1 species in the flowthrough decreases by about 1-4% after the AEX mixed-mode chromatography.

13. The method of claim 5, wherein the loading operating condition has a conductivity of 2-20 mS/cm.

14. The method of claim 5, wherein the loading operating condition has a conductivity of 10-15 mS/cm.

15. The method of claim 5, wherein the loading operating condition has a pH of about 5.5 to about 7.5.

16. The method of claim 5, wherein the loading operating condition has a pH of about 6.2 to about 7.2.

17. The method of claim 5, wherein the loading operating condition has a conductivity of 8-14 mS/cm, and a pH of about 7.0 to 7.4.

18. The method of claim 5, wherein the loading operating condition has a pH of about 6.0 to 7.5, and a conductivity of about 2-15 mS/cm.

19-20. (canceled)

21. The method of claim 5, wherein the load challenge is about 400 g/L of anti-PD-1 antibodies.

22. The method of claim 5, wherein the loading operation condition comprises a salt selected from the group consisting of sodium chloride, sodium acetate, sodium phosphate, and Tris-HCl.

23. The method of claim 22, wherein the salt is sodium phosphate and/or sodium chloride.

24. The method of claim 5, wherein the mixed-mode AEX provides one or more of a hydrophobic, hydrophilic, hydrogen bonding and thiophilic interaction; and an ionic interaction with the anti-PD-1 antibodies.

25. The method of claim 15, wherein the mixed-mode AEX provides at least a hydrophobic interaction; and an ionic interaction with the anti-PD-1 antibodies.

26. The method of claim 5, wherein the resin of the mixed-mode AEX comprises a positively charged aromatic compound.

27. The method of claim 15, wherein the resin of the mixed-mode AEX comprises N-benzyl-n-methyl ethanolamine attached.

28. (canceled)

29. The method of claim 15, wherein the mixed-mode AEX resin comprises 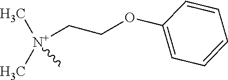
attached to the resin.

30. (canceled)

31. The method of claim 5, wherein the load fluid is an eluate from a protein A chromatography performed in bind and elute mode followed by AEX chromatography performed in flowthrough mode.

32-35. (canceled)

36. A composition of anti-human PD-1 antibodies or antigen-binding fragments obtainable by the method of claim 5.

37. (canceled)

38. (canceled)

39. The method of claim 10, wherein the loading operating condition has a conductivity of 2-20 mS/cm.

40. The method of claim 10, wherein the loading operating condition has a pH of about 5.5 to about 7.5.

41. The method of claim 40, wherein the mixed-mode AEX provides at least a hydrophobic interaction; and an ionic interaction with the anti-PD-1 antibodies.

42. The method of claim 40, wherein the resin of the mixed-mode AEX comprises a positively charged aromatic compound.

43. The method of claim 41, wherein the resin of the mixed-mode AEX comprises N-benzyl-n-methyl ethanolamine attached.

44. The method of claim 41, wherein the mixed-mode AEX resin comprises 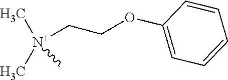
attached to the resin.


SEQUENCE LISTING (15 sequences)

SEQ ID NO: 1 - protein, 15 aa, synthetic construct
RASKGVSTSGYSYLH

SEQ ID NO: 2 - protein, 7 aa, synthetic construct
LASYLES

SEQ ID NO: 3 - protein, 9 aa, synthetic construct
QHSRDLPLT

SEQ ID NO: 4 - protein, 111 aa, synthetic construct [sequence omitted]

SEQ ID NO: 5 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
NYYMY

SEQ ID NO: 7 - protein, 17 aa, synthetic construct
GINPSNGGTNFNEKFKN

SEQ ID NO: 8 - protein, 11 aa, synthetic construct
RDYRFDMGFDY

SEQ ID NO: 9 - protein, 120 aa, synthetic construct [sequence omitted]

SEQ ID NO: 10 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 446 aa, synthetic construct
  SITE 1: note = Pyroglutamate
XVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 447 aa, synthetic construct
  SITE 1: note = Pyroglutamate
XVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 446 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 14 - protein, 445 aa, synthetic construct
  SITE 1: note = Pyroglutamate
XVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 15 - protein, 445 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSL